(19) United States
(12) Patent Application Publication
(10) Pub. No.: US 2019/0212290 A1
(43) Pub. Date: Jul. 11, 2019

(54) SYSTEMS AND METHODS FOR ELECTROCHEMICAL CREATININE ASSAYS AND BLOOD UREA NITROGEN

(71) Applicant: POLYMER TECHNOLOGY SYSTEMS, INC., Indianapolis, IN (US)

(72) Inventors: Brittney Werner, Fairland, IN (US); Gary Hughes, Camby, IN (US)

(21) Appl. No.: 16/245,063

(22) Filed: Jan. 10, 2019

Related U.S. Application Data
(60) Provisional application No. 62/616,339, filed on Jan. 11, 2018.

(51) Int. Cl. G01N 27/327; C12N 9/04; G01N 33/543

(52) U.S. Cl. CPC G01N 27/3272; C12N 9/0006; C12Y 101/01118; G01N 2333/902; G01N 33/5438

(57) ABSTRACT

A system for the electrochemical detection of analyte levels includes a test strip including an electrode and a counter electrode, the electrode and counter electrode located proximate to a sample reception area. The system further includes a coating on one of the electrode and counter electrode, the coating including a reagent coating for an analyte. The reagent coating includes a creatinine iminohydrolase, deamido NAD⁺, ATP, and NAD Synthetase/Mg²⁺.

### CROSS-REFERENCES TO RELATED APPLICATIONS

This application claims the benefit of U.S. Provisional application No. 62/616,339 filed Jan. 11, 2018, which is incorporated herein by reference in its entirety.

### BACKGROUND

Creatine (C₄H₉O₂N₃ or α-methyl guanidine-acetic acid) is a compound present in vertebrate muscle tissue, principally as phosphocreatine. Creatine is synthesized primarily in the liver and also in the pancreas and the kidneys. Creatine helps produce energy needed to contract muscles, and it is produced at a relatively constant rate. Creatine eventually is spontaneously degraded into creatinine by muscle and is released into the blood. It then is excreted by the kidneys and removed by the body by glomerular filtration.

The amount of creatinine produced is relatively stable in a given person. Serum creatinine level, therefore, is determined by the rate it is being removed, which is roughly a measure of kidney function. If kidney function falls, serum creatinine levels will rise. Thus, blood levels of creatinine are a good measure of renal function. Usually, increased creatinine levels do not appear unless significant renal impairment exists.

According to the American Diabetes Association (ADA), 20% to 30% of patients with diabetes develop diabetic kidney disease (nephropathy). Further, some authorities recommend measurement of serum creatinine levels in non-diabetic patients to screen for renal dysfunction because of increasing evidence that dietary protein restriction and use of angiotensin-converting enzyme (ACE) inhibitors can retard progression once renal insufficiency develops. Thus, the need for creatinine testing as a measure of kidney function is well established.

Additionally, another measure of kidney related health, relates to the concentration of blood urea nitrogen (BUN). In many scenarios, it is useful to report a ratio to creatinine to blood urea nitrogen. Therefore, having complementary tests for both analytes would be useful.

### BRIEF SUMMARY

In one embodiment, a method for detecting a blood analyte includes producing ammonia (NH₃) from a first reaction and reacting the ammonia with deamido NAD⁺, ATP, and NAD Synthetase/Mg²⁺ to produce NAD⁺. The method further includes measuring a level of the blood anaylte with at least two electrodes. Alternatively, the NAD⁺ is reacted with a dehydrogenase in order to perform the measuring. In one alternative, the dehydrogenase is glucose dehydrogenase. In another alternative, diaphorase and a mediator are further used in the measuring. Alternatively, the blood analyte is creatinine and the first reaction includes reacting the creatinine with creatinine iminohydrolase. In another alternative, the blood analyte is urea and the first reaction includes reacting the urea with urease.

In one embodiment, a system for the electrochemical detection of analyte levels includes a test strip including an electrode and a counter electrode, the electrode and counter electrode located proximate to a sample reception area. The system further includes a coating on one of the electrode and counter electrode, the coating including a reagent coating for an analyte. In one alternative, the reagent coating includes a creatinine iminohydrolase, deamido NAD⁺, ATP, and NAD Synthetase/Mg²⁺. Alternatively, the reagent coating includes glucose, glucose dehydrogenase, diaphorase, and a mediator. In another alternative, the mediator is selected from the list consisting of methylene blue, meldora blue, phenazine methosulfate, 2,6-Diclorophenol indophenol, nile blue, and potassium ferricyanide. Alternatively, the reagent coating includes a urease, deamido NAD⁺, ATP, and NAD Synthetase/Mg²⁺. In another alternative, the reagent coating includes a surfactant and a buffer. Alternatively, the reagent buffer includes a binder and a stabilizer.

In one embodiment, a system for the electrochemical detection of analyte levels includes a test strip including an electrode and a counter electrode, the electrode and counter electrode located proximate to a sample reception area. The system further includes a coating on one of the electrode and counter electrode, the coating including a reagent coating for an analyte. The system further includes an analyzer for receiving the test strip and including instructions stored on a non-transitory medium for applying a current to the test strip and responsively determining an amount of the analyte. In one alternative, the reagent coating includes a creatinine iminohydrolase, deamido NAD⁺, ATP, and NAD Synthetase/Mg²⁺Alternatively, the reagent coating includes glucose, glucose dehydrogenase, diaphorase, and a mediator. In another alternative, the mediator is selected from the list consisting of methylene blue, meldora blue, phenazine methosulfate, 2,6-Diclorophenol indophenol, nile blue, and potassium ferricyanide. Alternatively, the reagent coating includes a urease, deamido NAD⁺, ATP, and NAD Synthetase/Mg²⁺.

In one embodiment, a method of detecting an analyte includes providing an electrochemical test strip. The method further includes placing the electrochemical test strip in an analyzer. The method further includes placing a blood sample on the electrochemical test strip. The method further includes measuring a current provided through the blood sample and the electrochemical test strip. The method further includes calculating a level of an analyte, the analyte selected from the group consisting of creatinine and urea, with the analyzer based on the current. In one alternative, the test strip includes an electrode and a counter electrode, the electrode and counter electrode located in a sample reception area; and a coating on one of the electrode and counter electrode, the coating including a reagent coating for creatinine and the method further includes reacting the creatinine with creatinine iminohydrolase. Alternatively, the method further includes producing ammonia (NH₃) from a first reaction; and reacting the ammonia with deamido NAD⁺, ATP, and NAD Synthetase/Mg²⁺ to produce NAD⁺. Alternatively, the NAD⁺ is reacted with a dehydrogenase in order to perform the measuring. In another alternative, the dehydrogenase is glucose dehydrogenase. Alternatively, diaphorase and a mediator are further used in the measuring. In another alternative, the test strip includes an electrode and a counter electrode, the electrode and counter electrode located in a sample reception area; and a coating on one of the electrode and counter electrode, the coating including a reagent coating for urea and the method further includes reacting the urea with urease.

### BRIEF DESCRIPTION OF THE DRAWINGS

FIG. 1 shows a proof-of-concept graph that was produced using whole blood and the proposed creatinine reagent scheme;

FIG. 2 shows one embodiment of an electrochemical reaction scheme for creatinine;

FIG. 3 shows one embodiment of an electrochemical reaction scheme for BUN; and

FIG. 4 shows one embodiment of the strip design.

### DETAILED DESCRIPTION

Certain terminology is used herein for convenience only and is not to be taken as a limitation on the embodiments of the systems and methods for electrochemical creatinine assays. In the drawings, the same reference letters are employed for designating the same elements throughout the several figures. In many embodiments, systems and methods for electrochemical creatinine assays includes the use of four enzymes. In many embodiments, the reaction scheme is based on the formation of ammonia. Ammonia is created by the usage of creatinine iminohydrolase or deiminase enzyme. Ammonia is then processed to be readable in an electrochemical format.

In many embodiments, the system is designed to create an electrochemical creatinine/BUN assay. In order for embodiments to be useful in the imaging market, a high level of precision is required to determine the difference between 1 and 1.1 mg/dL creatinine. This level of precision is difficult to achieve with a reflectance based test. Since electrochemical assays generally have better precision, this approach is used for creatinine.

The proposed test has many potential advantages. First, electrochemical test strips are generally inexpensive to produce due to the automation and small amounts of reagent used. Second, the disclosed electrochemical creatinine/BUN assay is not dependent on oxygen and thus can test both venous and capillary blood. Third, testing creatinine via electrochemistry generally results in better precision. Precision and accuracy are key if this assay is to be developed for the imaging markets. Precision is also aided by having four enzyme reactions instead of five. Fourth, the test range of an electrochemical creatinine/BUN assay, in many embodiments, is larger than a reflectance assay. Reflectance tests are limited at the high concentrations by the amount of color that can be generated. However, electrochemical assays are able to measure much higher concentrations. Fifth, in many embodiments the sample size is small; at 2-5 μL instead of 20 μL.

In many embodiments, a scheme for electrochemically detecting creatinine is used. Creatinine is a waste molecule from muscle metabolism. The bloodstream transports creatinine to the kidneys where the majority of it is filtered out and disposed as urine. Elevated creatinine levels are an indication of kidney malfunction. Creatinine is an important test to determine the functionality of the kidneys and can be used in the imaging markets to determine if contrast dye should be given to a patient.

There are several methods to measure creatinine. The most popular enzymatic method is the reaction scheme listed below.

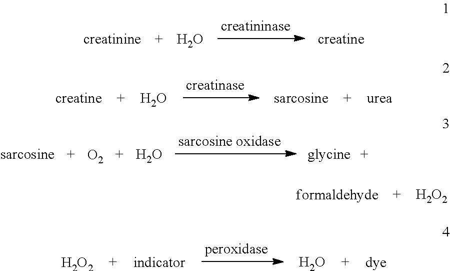

For this enzymatic method, endogenous creatine will be an interferant causing over recovery unless it is removed. Chemistry analyzers that use the above method are able to remove the influence of endogenous creatine by having two assays and subtracting the creatine out. A POC device would need to also subtract the native creatine influence. This doubles the cost of creating a POC creatinine assay, and there could be compounded error based on the subtraction step.

A more direct reaction scheme for a POC creatinine assay is listed in the equations below. This is the method that PTS Diagnostics currently employs under US Patent Pub. No. 2004/0126833, which is hereby incorporated by reference. This is a complex reaction with 5 different enzymes. It is also fairly expensive due to the enzyme costs. Additionally, the NMHase and CSHase have very low activities requiring a large mass of enzymes.

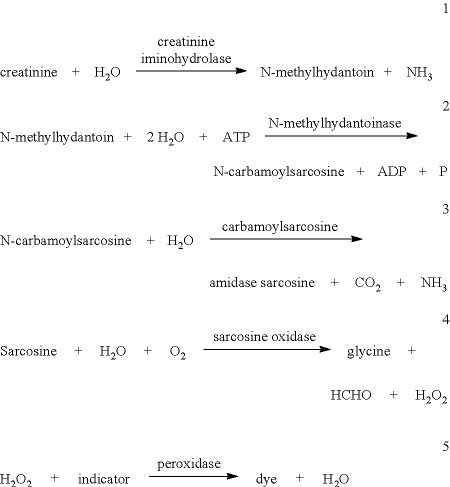

Therefore, in many embodiments, it is desirable to employ a better scheme. A better method to measure creatinine for an electrochemical POC system involves using only four enzymes. The reaction scheme is described below and shown in FIG. 2.

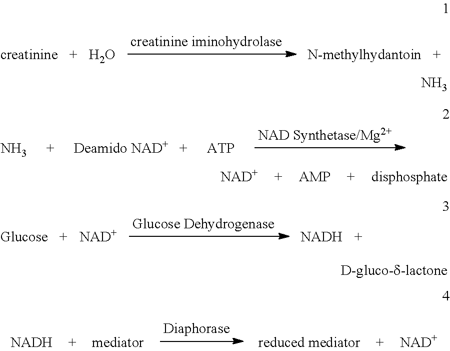

This reaction scheme is based on the formation of NH3 (ammonia) by the creatinine iminohydrolase or deiminase enzyme. The normal range of ammonia in the blood is 11-32 μM/L. Normal creatinine blood levels will range from 74-107 μM/L. The interference from ammonia may be minimal or it may need to be subtracted out. If the endogenous ammonia needs to be subtracted from the assay, it would be as simple as only using the reactions from step 2-4 in a separate channel. The POC analyzer could also report ammonia levels if desired. High ammonia levels would be an indication of cirrhosis of the liver or hepatitis.

The glucose/glucose dehydrogenase reaction in reaction steps 3-4 is not the only reaction that could be used in this assay. In various alternatives, any substrate that reacts with NAD⁺ and a dehydrogenase can be used. Therefore, the glucose may be substituted out. The assay is essentially measuring the amount of NAD⁺ formed from step 2. For example, one could use β-hydroxybutyrate and β-hydroxybutyrate dehydrogenase to measure the NAD⁺ formed. Glucose dehydrogenase will probably be chosen based on availability and cost.

An advantage of the reaction scheme described above is that the NAD⁺ is amplified by the enzyme cycling system. Notice that NAD⁺ is also produced at the end of reaction 4. This is helpful in assays like creatinine where the analyte levels are very low. Another way to boost signal is by using interdigitated electrodes.

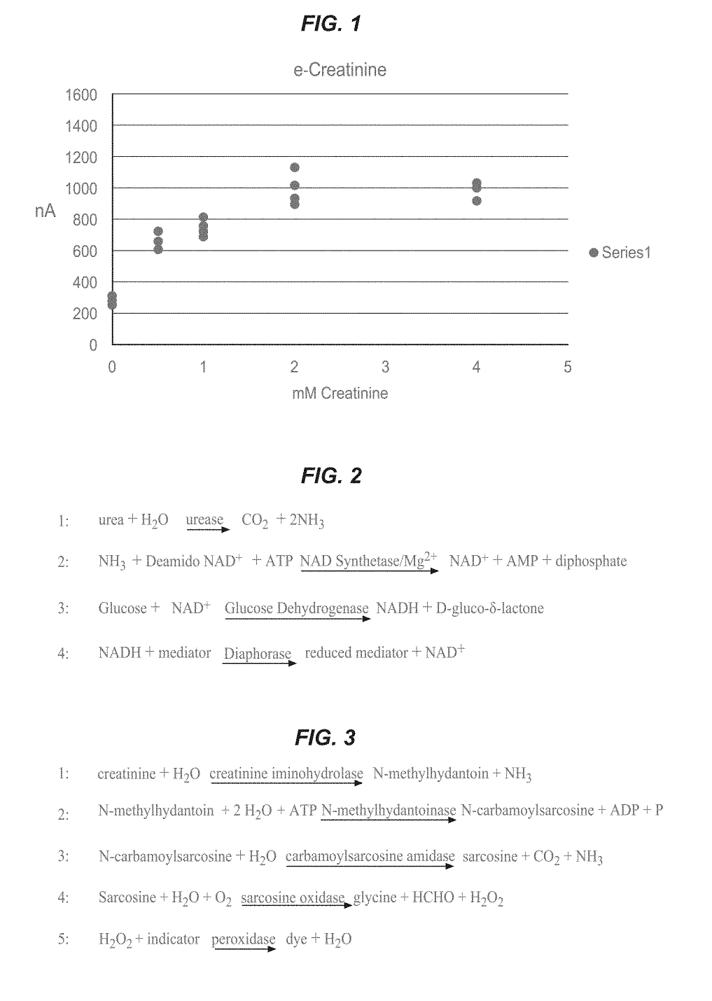

Reaction steps 3-4 are well characterized for an electrochemical reaction. Any suitable mediator that will create an excellent dose response will work. For our purposes, we chose ferricyanide to show proof of concept. Without optimizing any components, a reagent was made and dried down on carbon electrodes. Creatinine samples were made in saline with 20 mM glucose to facilitate the reaction. The results are in FIG. 1. FIG. 1 shows electrochemical response for creatinine. Carbon sensors were used with 400 mV potential. While the reagent has not been optimized, the workings of a creatinine assay are clear. The signal can be amplified with interdigitated electrodes.

In addition to having an amperometric creatinine sensor, other assays may be incorporated using a versatile electrochemical test strip and offer multiple tests with the creatinine test. While the creatinine is tested, it may be helpful to check other important analytes such as urea, ammonia, glucose, ketones, triglycerides etc.

In another embodiment, a similar reaction scheme may be used to determine BUN (blood urea nitrogen). In many embodiments, this test is combined with the creatinine assay on a versatile electrochemical test strip. In addition to creatinine, many clinicians would like to know the urea or BUN levels for evaluating kidney function. Often a BUN to creatinine ratio is reported.

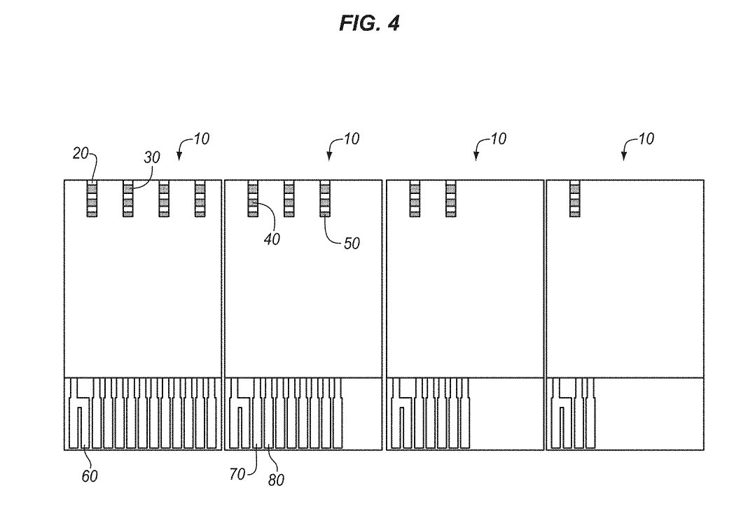

The reaction scheme proposed for creatinine will work for urea as well, with some modifications. Reacting urea with urease will also produce ammonia. The urea is less likely to be influenced by ammonia interference as the normal range of urea is 0.64 to 2.53 mM. The reaction scheme is also shown in FIG. 3.

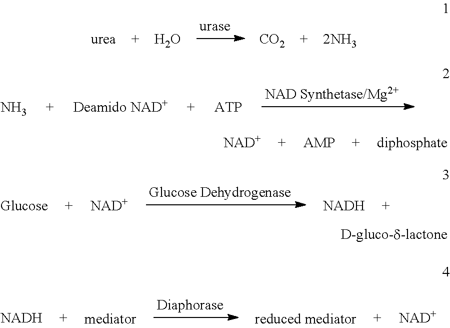

In summary, an electrochemical creatinine and BUN assay may be created using the above reaction scheme for creatinine. It may also be possible to create an ammonia assay if the sensitivity can be reached. The advantages of using the proposed reaction scheme is that the reaction scheme for the last three reactions is the same for both BUN and creatinine. Also, both assays will be immune to oxygen so both venous and capillary blood can be used. This will be a desired test for investigating kidney function.

In many embodiments, gold or carbon sensors (electrodes) may be used. Alternatively, platinum, silver chloride, or other types of electrodes may be used. An advantage of gold sensors is having less background signal while maintaining the same slope. Using gold sensors would also be advantageous for methods to measure hematocrit by AC impedance based on techniques that include the usage of phase angle shift in order to detect hematocrit.

In addition to having an amperometric creatinine sensor, a versatile electrochemical test strip may offer multiple tests with the creatinine test. While the creatinine is tested, it may be helpful to check other important analytes such as glucose, ketones, triglycerides, etc. In some embodiments, an electrochemical sensor may include multiple testing areas as shown in FIG. 4.

FIG. 4 shows one embodiment of the strip design. Shown are four strips 10. From left to right, the strips 10 have 4, 3, 2, and 1 sample receiving ports 20. Each sample receiving port may have an electrode 30, a counter electrode 40, and a reference electrode 50. The reference electrode 50 may provide for a fill indication, as it will only pass a voltage when the sample reaches the electrode 50. The contacts 70, 80 also are visible, which interconnect with the electrodes and connect to contacts in the analyzer when inserted. The strip size does not change depending on the number of assays. In addition, the electrode placement does not change depending on the type of assays. Depending on what is desired for the testing scheme, sheets are printed for one, two, three, or four analytes. The spirit behind this is not to limit the size of the panel to only four analytes, but to provide a concept that is protected whether one or ten analytes are tested. Also, the electrodes do not all need to be on one side of the strip. Superior technology may be able to place electrodes on both sides of the strip, thus allowing for miniaturization. These test strips are designed to fit with corresponding meters for measuring the blood analyte levels.

In some embodiments, single analyte test strips are designed to have the same location with at least four associated electrodes. The electrode 60 that appears as an “h” is used for strip detection by the analyzer. The remaining assays will have at least three electrodes—one for sample fill detection, and the other two as a counter electrode and a working electrode. These assays are not limited to a set number of electrodes, for it is foreseen in some embodiments that more electrodes may be added for purposes of determining and correcting for hematocrit or other interfering substances.

In multiple configurations, reagents may be painted on the electrodes. Alternatively, reagents may be printed, coated, dip coated, or otherwise applied, as will be apparent in the field. Various types of electrodes may be used as well, including those made of carbon, gold, platinum, copper, or other conductive materials, as will be apparent to those in the field.

FIG. 4 displays separate blood sampling ports for each assay. Some embodiments may include separate sampling ports, particularly if there could be “cross talk” between reagents. In summary, embodiments of a novel idea for an electrochemical creatinine sensor have been presented. An electrochemical creatinine test will have a smaller sample size, shorter test time, better precision, and will be cheaper to manufacture and may easily be combined with a BUN assay.

In one embodiment, a test strip and meter combination is provided. The test strip includes test areas for creatinine and urea. The test strip and meter combination, upon the addition of a sample, tests for creatinine and urea and produces a ratio of the two as well as individual measurements of each.

In many embodiments, parts of the system are provided in devices including microprocessors. Various embodiments of the systems and methods described herein may be implemented fully or partially in software and/or firmware. This software and/or firmware may take the form of instructions contained in or on a non-transitory computer-readable storage medium. Those instructions then may be read and executed by one or more processors to enable performance of the operations described herein. The instructions may be in any suitable form such as, but not limited to, source code, compiled code, interpreted code, executable code, static code, dynamic code, and the like. Such a computer-readable medium may include any tangible non-transitory medium for storing information in a form readable by one or more computers such as, but not limited to, read only memory (ROM); random access memory (RAM); magnetic disk storage media; optical storage media; a flash memory, etc.

Embodiments of the systems and methods described herein may be implemented in a variety of systems including, but not limited to, smartphones, tablets, laptops, and combinations of computing devices and cloud computing resources. For instance, portions of the operations may occur in one device, and other operations may occur at a remote location, such as a remote server or servers. For instance, the collection of the data may occur at a smartphone, and the data analysis may occur at a server or in a cloud computing resource. Any single computing device or combination of computing devices may execute the methods described.

While specific embodiments have been described in detail in the foregoing detailed description and illustrated in the accompanying drawings, it will be appreciated by those skilled in the art that various modifications and alternatives to those details could be developed in light of the overall teachings of the disclosure and the broad inventive concepts thereof. It is understood, therefore, that the scope of this disclosure is not limited to the particular examples and implementations disclosed herein but is intended to cover modifications within the spirit and scope thereof as defined by the appended claims and any and all equivalents thereof. Note that, although particular embodiments are shown, features of each attachment may be interchanged between embodiments.

What is claimed as new and desired to be protected by Letters Patent of the United States is:

1. A system for the electrochemical detection of an analyte level, the system comprising:
a test strip including an electrode and a counter electrode, the electrode and counter electrode located proximate to a sample reception area; and
a coating on one of the electrode and counter electrode, the coating including a reagent coating for an analyte.

2. The system of claim 1, wherein the reagent coating includes a creatinine iminohydrolase, deamido NAD⁺, ATP, and NAD Synthetase/Mg²⁺.

3. The system of claim 2, wherein the reagent coating includes glucose, glucose dehydrogenase, diaphorase, and a mediator.

4. The system of claim 3, wherein the mediator is selected from the list consisting of methylene blue, meldora blue, phenazine methosulfate, 2,6-Diclorophenol indophenol, nile blue, and potassium ferricyanide.

5. The system of claim 1, wherein the reagent coating includes a urease, deamido NAD⁺, ATP, and NAD Synthetase/Mg²⁺.

6. The system of claim 5, wherein the reagent coating includes a surfactant and a buffer. The system of claim 6, wherein the reagent buffer includes a binder and a stabilizer.

8. A system for the electrochemical detection of analyte levels, the system comprising:
a test strip including an electrode and a counter electrode, the electrode and counter electrode located proximate to a sample reception area;
a coating on one of the electrode and counter electrode, the coating including a reagent coating for an analyte; and
an analyzer for receiving the test strip and including instructions stored on a non-transitory medium for applying a current to the test strip and responsively determining an amount of the analyte.

9. The system of claim 8, wherein the reagent coating includes a creatinine iminohydrolase, deamido NAD⁺, ATP, and NAD Synthetase/Mg²⁺.

10. The system of claim 9, wherein the reagent coating includes glucose, glucose dehydrogenase, diaphorase, and a mediator.

11. The system of claim 10, wherein the mediator is selected from the list consisting of methylene blue, meldora blue, phenazine methosulfate, 2,6-Diclorophenol indophenol, nile blue, and potassium ferricyanide.

12. The system of claim 8, wherein the reagent coating includes a urease, deamido NAD⁺, ATP, and NAD Synthetase/Mg²⁺.

13. A method for detecting a blood analyte, the method comprising:
producing ammonia (NH3) from a first reaction;
reacting the ammonia with deamido NAD⁺, ATP, and NAD Synthetase/Mg²⁺ to produce NAD⁺; and
measuring a level of the blood anaylte with at least two electrodes.

14. The method of claim 13, wherein the NAD⁺ is reacted with a dehydrogenase in order to perform the measuring.

15. The method of claim 14, wherein the dehydrogenase is glucose dehydrogenase.

16. The method of claim 15, wherein diaphorase and a mediator are further used in the measuring.

17. The method of claim 13, wherein the blood analyte is creatinine and the first reaction include reacting the creatinine with creatinine iminohydrolase.

18. The method of claim 13, wherein the blood analyte is urea and the first reaction include reacting the urea with urease.

19. A method of detecting an analyte, the method comprising:
providing an electrochemical test strip;
placing the electrochemical test strip in an analyzer;
placing a blood sample on the electrochemical test strip;
measuring a current provided through the blood sample and the electrochemical test strip; and
calculating a level of an analyte, the analyte selected from the group consisting of creatinine and urea, with the analyzer based on the current.

20. The method of claim 19, wherein the test strip includes an electrode and a counter electrode, the electrode and counter electrode located in a sample reception area; and a coating on one of the electrode and counter electrode, the coating including a reagent coating for creatinine and the method further includes reacting the creatinine with creatinine iminohydrolase.

21. The method of claim 20, wherein the method further includes
producing ammonia (NH₃) from a first reaction; and
reacting the ammonia with deamido NAD⁺, ATP, and NAD Synthetase/Mg²⁺ to produce NAD⁺.

22. The method of claim 21, wherein the NAD⁺ is reacted with a dehydrogenase in order to perform the measuring.

23. The method of claim 22, wherein the dehydrogenase is glucose dehydrogenase.

24. The method of claim 23, wherein diaphorase and a mediator are further used in the measuring.

25. The method of claim 19, wherein the test strip includes an electrode and a counter electrode, the electrode and counter electrode located in a sample reception area; and a coating on one of the electrode and counter electrode, the coating including a reagent coating for urea and the method further includes reacting the urea with urease.
